(19) United States
(12) Patent Application Publication
(10) Pub. No.: US 2022/0344009 A1
(43) Pub. Date: Oct. 27, 2022

(54) Systems and Methods for Designing Efficient Randomized Trials Using Semiparametric Efficient Estimators for Power and Sample Size Calculation

(71) Applicant: Unlearn.AI, Inc., San Francisco, CA (US)

(72) Inventor: Alejandro Schuler da Costa Ferro, San Francisco, CA (US)

(73) Assignee: Unlearn.AI, Inc., San Francisco, CA (US)

(21) Appl. No.: 17/659,428

(22) Filed: Apr. 15, 2022

Related U.S. Application Data
(60) Provisional application No. 63/176,111, filed on Apr. 16, 2021.

(51) Int. Cl. G16H 10/20

(52) U.S. Cl. CPC G16H 10/20

(57) ABSTRACT

Systems and method for designing efficient randomized trials using semiparametric efficient estimators for power and sample size calculation in accordance with embodiments of the invention are illustrated. One embodiment includes a method for sample size estimation using semiparametric efficient estimators. The method includes generating sets of one or more subject characteristics of a plurality of trial subjects based on data of prior trials and registry data, estimating sets of one or more population parameters based on the sets of one or more subject characteristics, estimating asymptotic variances of a plurality of estimators using the sets of one or more population parameters, setting a desired power level for the trial, and determining a sample size necessary to attain the desired power level for the trial based on the asymptotic variances and a treatment effect estimated by a semiparametric efficient estimator.

### CROSS-REFERENCE TO RELATED APPLICATIONS

The current application claims the benefit of and priority under 35 U.S.C. § 119(e) to U.S. Provisional Patent Application No. 63/176,111 entitled “Designing Efficient Randomized Trials: Power and Sample Size Calculation When Using Semiparametric Efficient Estimators” filed Apr. 16, 2021. The disclosure of U.S. Provisional Patent Application No. 63/176,111 is hereby incorporated by reference in its entirety for all purposes.

### FIELD OF THE INVENTION

The present invention generally relates to clinical trial design and analysis, and, more specifically, using semiparametric efficient estimators to estimate sample size for clinical trials.

### BACKGROUND

Clinical research and clinical trials aim to study the safety and efficacy of biomedical or behavioral interventions on humans. When new drugs and medical devices are invented, they must undergo rigorous trials to generate data on its dosage and safety in order to approved by the relevant authorities for clinical use. Test articles that do not produce satisfactory safety or efficacy levels will not be approved for mass commercial use.

Randomized trials are one method used to conduct a clinical trial. In clinical research, a randomized trial generally has two arms, namely the treatment arm and the control arm. Trials compare a proposed new treatment represented by the treatment arm against an existing treatment represented by the control arm to determine the efficacy of the new treatment. When no generally accepted existing treatments are available, a placebo treatment may be used in place of the existing treatment. A well-designed randomized trial may provide reliable indication on not only the trial outcome, but also information on possible adverse effects of the experiment.

An estimator is a rule for estimating the value of a certain estimand based on observed data. Estimators are an important tool in trial design, as researchers use various estimators to predict required parameters associated with the trial in order to design a robust trial. Among estimators that are unbiased (i.e. estimators that produce estimates that are correct on average), an estimator is deemed to be more accurate if it has a smaller asymptotic variance, that is, if the estimator produces estimated values that are closest to the true value.

### SUMMARY OF THE INVENTION

Systems and method for designing efficient randomized trials using semiparametric efficient estimators for power and sample size calculation in accordance with embodiments of the invention are illustrated. One embodiment includes a method for sample size estimation using semiparametric efficient estimators, where the method includes generating sets of one or more subject characteristics of a plurality of trial subjects based on data of prior trials and registry data. The method further includes estimating sets of one or more population parameters based on the sets of one or more subject characteristics, and estimating asymptotic variances of a plurality of estimators using the sets of one or more population parameters. The method further includes setting a desired power level for the trial, and determining a sample size necessary to attain the desired power level for the trial based on the asymptotic variances and a treatment effect estimated by a semiparametric efficient estimator.

In another embodiment, the method includes steps for estimating the treatment effect using the semiparametric efficient estimator, where in estimating the treatment effect includes estimating a conditional means function in a treatment group based on sets of one or more subject characteristic data, deriving an estimate of marginal means based on the sets of one or more subject characteristics and the conditional means function, and estimating a treatment effect based on the marginal means.

In a further embodiment, the method further includes steps for estimating the conditional means function includes splitting the sets of one or more subject characteristic data into a plurality of overlapping folds, fitting a corresponding machine learning model for each of the plurality of overlapping folds, excluding subject characteristic data of a last of the plurality of folds, and training the machine learning model to estimate the conditional means function by predicting subject characteristic data of the last of the plurality of folds.

In still another embodiment, the sets of one or more subject characteristics include outcomes, baseline covariates, and treatment assignments.

In a still further embodiment, the semiparametric efficient estimator is an augmented inverse propensity weighting (AIPW) estimator.

In yet another embodiment, the sets of one or more population parameters can be estimated with a machine learning model in combination with the sets of one or more subject characteristics.

In a yet further embodiment, the sets of one or more population parameters include marginal variances, average conditional variances, and a correlation between conditional means.

In another additional embodiment, the semiparametric efficient estimator is a targeted maximum likelihood estimation (TMLE) estimator.

One embodiment includes a non-transitory machine readable medium containing processor instructions for sample size estimation using semiparametric efficient estimators, where execution of the instructions by a processor causes the processor to perform a process that includes, generating sets of one or more subject characteristics of a plurality of trial subjects based on data of prior trials and registry data, estimating sets of one or more population parameters based on the sets of one or more subject characteristics, estimating asymptotic variances of a plurality of estimators using the sets of one or more population parameters, setting a desired power level for the trial, and determining a sample size necessary to attain the desired power level for the trial based on the asymptotic variances and a treatment effect estimated by a semiparametric efficient estimator.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a flow chart of a process to estimate a sample size necessary to attain a desired power of a trial using semiparametric estimators.

FIG. 2 is a flow chart of a process for estimating a treatment effect using an augmented inverse propensity weighting (AIPW) estimator in accordance with an embodiment of the invention.

FIG. 3 illustrates simulation results of the necessary sample size to attain a desired power level of a trial using various estimators under different scenarios.

FIG. 4 illustrates simulation results of type I error rates of various estimators when estimating the sample size necessary to attain a desired power level of a trial under different scenarios.

FIG. 5 is a high-level block diagram of a system for an estimation process to be implemented on in accordance with an embodiment of the invention.

FIG. 6 is a high-level block diagram of an application that executes an estimation process in accordance with an embodiment of the invention.

FIG. 7 is a diagram of a network where an estimation process may be implemented on in accordance with an embodiment of the invention.

### DETAILED DESCRIPTION

Turning now to the drawings, systems and methods for designing efficient randomized trials using semiparametric efficient estimators for power and sample size calculation are illustrated. Clinical research aims to estimate the effect of a new treatment, and to make sure that the new treatment is safe. Researchers perform clinical trials of various treatments in an effort to ascertain the effect of the treatments. In general, randomized clinical trials are utilized to a great effect with how randomization cancels out the effects of potentially unobserved confounders in expectation.

Randomized clinical trials often require a sufficiently large sample size for the estimated result to be representative. However, with a larger sample size, the natural variability of the sample also increases, making the treatment estimates uncertain. The power of a trial is defined as the likelihood that the trial is able to positively identify an effect of a certain size. The degree of uncertainty negatively affects the power of the trial, and therefore it is standard to design a trial that would yield a power over 80% to minimize likelihood of failure.

Factors affecting power include characteristics of the data-generating process, the aggressiveness of the rule used to determine effect, the number of subjects enrolled into the trial, and the method of data analysis. As a matter of trial design, the number of subjects enrolled in the trial needs to be determined before trial has even begun. Traditionally, this determination was performed with the assumption of an unadjusted analysis using unadjusted estimators which led to conservative sample sizes at a higher cost.

All estimators have a sampling variance. The smaller the sampling variance of an estimator, the more power a trial will have. An estimator is also more efficient with a smaller sampling variance. The most efficient estimators are semiparametric efficient estimators. Semiparametric efficient estimators are able to keep its asymptotic sampling variance low, which produces maximum trial power while keeping its type I error rate low to control false positive rates. The resulting confidence intervals will also be as small as possible. Previously, semiparametric efficient estimators were mainly used in the analysis of trial data after the trial is complete. Embodiments of the invention aim to leverage the benefits of semiparametric efficient estimators in the designing of a trial, and to achieve an accurate estimation of the necessary sample size required for the trial to produce the desired power level while keeping the sample size small for lower costs and ease of data management.

In general, clinical trials consists of two arms, namely a control arm and a treatment arm. Trial subjects assigned to the control arm are generally given existing treatments or placebo, whereas trial subjects assigned to the treatment arm are given the new treatment being researched on. A comparison would then be done at the end of the trial to determine the efficacy of the new treatment.

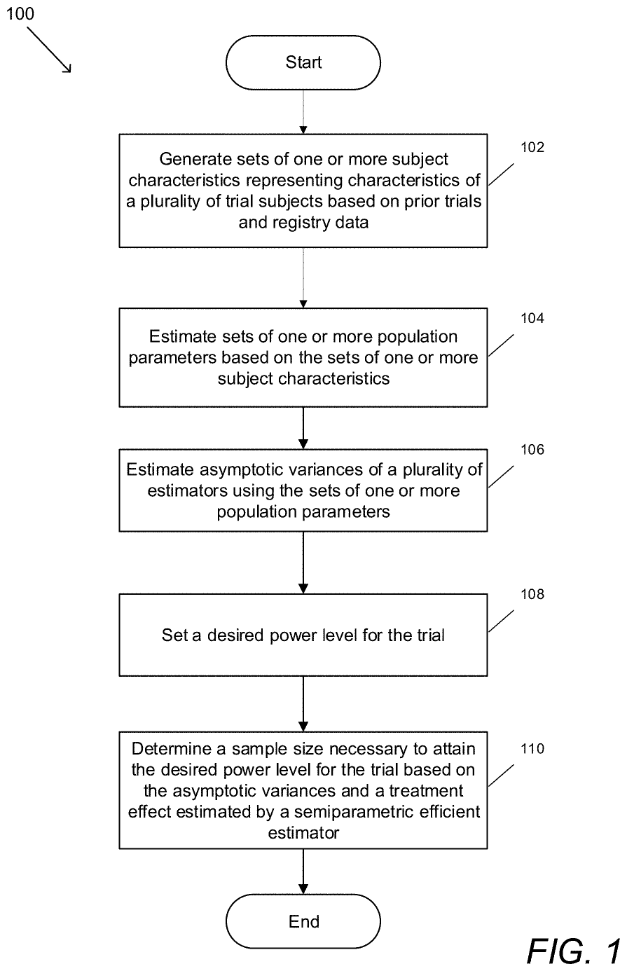

Turning now to FIG. 1, an estimation process 100 to estimate sample size necessary to attain a desired power of a trial using semiparametric estimators in accordance with an embodiment of the invention is illustrated. In many embodiments, process 100 generates (102) sets of one or more subject characteristics by sampling data from the control arm of prior trials and registry data, or by making prospective measurements for the trial subjects through methods including questionnaires, lab tests, and imaging. The sets of one or more subject characteristics include observed outcomes Yᵢ, baseline covariates Xᵢ and treatment assignment Wᵢ. The subject characteristics dataset is a set of n tuples (Xᵢ, Wᵢ, Yᵢ).

In accordance with embodiments of the invention, Y_{0,1} denotes the outcome that trial subject i would have obtained had they been assigned to the control arm, and Y_{1,i} denotes the outcome that trial subject i would have obtained had they been assigned to the treatment arm. The observed outcome Yᵢ corresponds to either Y_{0,i} or Y_{1,i} depending on which arm the trial subject is assigned to in reality. Additionally, let Y_{W}=WY₁+(1−W)Y₀. Taken together, process 100 structurally assumes that:

$$P ( X , W , Y , {Y}_{0} , {Y}_{1} ) = 1 ( Y = {Y}_{w} ) P ( W ) {\prod }_{i} P ( {X}_{i} {Y}_{0 , i} {Y}_{1 , i} ) \qquad ( 1 )$$

This means that the observed outcomes are the potential treatment outcomes corresponding to the assigned treatment, the treatment is assigned at random among the trial subjects, and the trial subjects are independent of each other. Trial subjects can also be assumed to be identically distributed.

In several embodiments of the invention, process 100 generates (102) subject characteristics using historical data of the treatment arm of previously already-existing trials conducted on the treatment of interest. Observed outcomes Yᵢ, baseline covariates Xᵢ, and treatment assignment Wᵢ are still considered under this generation scheme. Where treatment assignment W equals 0 in the embodiments described above as subject characteristics are sampled from prior control arm data, treatment assignment W equals 1 under this generation scheme as subject characteristics are sampled from historical data of treatment arms.

Process 100 in accordance with embodiments of the invention estimates (104) sets of one or more population parameters based on the sets of one or more subject characteristics. In several embodiments, the estimation (104) includes hypothesizing a bound for the population parameters. Population parameters may include marginal variances σ_{w}², average conditional variances κ_{w}², and a correlation between conditional means γ. Marginal variances in a clinical trial setting may be inferred from registry data, electronic health records, or prior studies on similar populations. Therefore, the variance is taken to be σ₀² as it is most often assumed that σ₀=σ₁ when there is a lack of reliable treatment arm data. Average conditional variances are estimated based on marginal variances. The upper bound of average conditional variances κ_{w}² may be estimated by averaging known marginal variances across sub-populations defined by the planned adjustment covariates. In some embodiments, a trial may be presumed to have an equal number of trial subjects of men and women where the biological sex is a baseline covariate subject to planned adjustment. The means of the marginal outcome variance among men and women would be a consistent estimator of an upper bound on the average conditional variance. The population can be arbitrarily divided as many times as existing data permits, so long as the manner in which the division is done is pre-specified. If only control arm data is available, the estimation would yield a κ₀².

In several embodiments, κ₀² may be estimated with a machine learning model in combination with subject characteristics. κ₀² is the Bayes mean-squared error (MSE) for estimating the expected treatment outcome conditioned on baseline covariates. Therefore, if there are existing data for treatment outcome and baseline covariates, a machine learning model may be trained using those data to produce a consistent estimator for an upper bound on the Bayes MSE since MSE is by definition the best possible model. Additionally, a usable upper bound may be determined even if a subset of the baseline covariates subject to planned adjustment were available.

The correlation between conditional means γ depends on the behavior of the treatment arm and is therefore unable to be estimated using subject characteristics based on control arm alone. However, in many embodiments, it is reasonable to assume that the treatment effect is additively constant across the population, which leads to γ=1 in these situations. Treatment effect, in a number of embodiments is represented by i=r(\hat{μ}₀,\hat{μ}₁), where function r defines the treatment effect from the true mean outcomes μ_{q}= [symbol] [Y_{q}]. A reasonable lower bound for γ would be greater than or equal to 0. In several embodiments, κ₀²=κ₁² may be assumed. In selected embodiments where treatment data is more readily available, no hypothesizing of population parameters may be necessary and all of σ₀², σ₁², κ₀², γ², and y may be available. In many embodiments of the invention, process 100 estimates (104) sets of one or more population parameters based on the sets of one or more subject characteristics generated using historical data of the treatment arm of previously already-existing trials conducted on the treatment of interest.

Process 100 in accordance with embodiments of the invention estimates (106) asymptotic variances of a plurality of estimators being used to design the trial based on the population parameters. Asymptotic variance measures how tight the estimated result is around the truth. A semiparametric efficient estimator is one that gives the smallest asymptotic variance, therefore it is necessary to examine the asymptotic variance of the estimators used in the process. In many embodiments of the invention, the estimator used is an augmented inverse propensity weighting (AIPW) estimator which will be explained in detail below. Let σ_{w}²≡ [symbol] [Y_{W}] be the marginal outcome variances in each treatment arm and κ_{W}²≡ [symbol] [ [symbol] [Y_{W}|X]] be the corresponding average conditional variances. The correlation between the conditional means is defined as γ=Corr[μ₀(X),μ₁(X)], and let

$${r}_{w}^{′} = \frac{\partial r}{\partial {μ}_{w}} ( {μ}_{0} , {μ}_{1} ) ,$$

the asymptotic variance of any semiparametric efficient estimator of the parameter τ=r(μ₀,μ₁) in many embodiments of the invention is:

$${\hat{v}}_{*}^{2} = {r}_{0}^{′ 2} ( \frac{{π}_{1}}{{π}_{0}} {\hat{κ}}_{0}^{2} + {\hat{σ}}_{0}^{2} ) + {r}_{1}^{′ 2} ( \frac{{π}_{0}}{{π}_{1}} {\hat{κ}}_{1}^{2} + {\hat{σ}}_{1}^{2} ) - 2 \vert {r}_{0}^{′} {r}_{1}^{′} \vert γ \sqrt{( {\hat{σ}}_{0}^{2} - {\hat{κ}}_{0}^{2} ) ( {\hat{σ}}_{1}^{2} - {\hat{κ}}_{1}^{2} )} \qquad ( 2 )$$

In several embodiments, conditional means functions μ_{w}(X)=μ_{w} are constants. This yields σ_{w}²=κ_{w}² and reduces equation (2) to

$${v}_{*}^{2} = {r}_{0}^{′2} \frac{{σ}_{0}^{2}}{{π}_{0}} + {r}_{1}^{′ 2} \frac{{σ}_{1}^{2}}{{π}_{1}} ,$$

which is the variance of an unadjusted (difference in means) estimator. This illustrates that the unadjusted estimator may be efficient when conditional means are constant because the covariates impart no exploitable information.

In some embodiments of the invention, other semiparametric efficient estimators could achieve the same efficiency and may be utilized in the process. The asymptotic variances of an AIPW and an unadjusted estimator are estimated according to the formula:

$${\hat{v}}_{AIPW}^{2} = {r}_{0}^{′ 2} ( \frac{{π}_{1}}{{π}_{0}} {\hat{κ}}_{0}^{2} + {\hat{σ}}_{0}^{2} ) + {r}_{1}^{′ 2} ( \frac{{π}_{0}}{{π}_{1}} {\hat{κ}}_{1}^{2} + {\hat{σ}}_{1}^{2} ) - 2 \vert {r}_{0}^{′} {r}_{1}^{′} \vert γ \sqrt{( {\hat{σ}}_{0}^{2} - {\hat{κ}}_{0}^{2} ) ( {\hat{σ}}_{1}^{2} - {\hat{κ}}_{1}^{2} )} \qquad ( 3 )$$

$${\hat{v}}_{unadj}^{2} = {r}_{0}^{′ 2} \frac{{\hat{σ}}_{0}^{2}}{{π}_{0}} + {r}_{1}^{′ 2} \frac{{\hat{σ}}_{1}^{2}}{{π}_{1}} \qquad ( 4 )$$

In a 1:1 randomized trial, π_{w}=½. Inclusion of an unadjusted estimator serves as a frame of comparison in the final determination of sample size necessary for the trial.

In another embodiment where π₁=π₀, σ₀=σ₁σ, and κ₀²=κ₁²=κ², \hat{ν}² is reduced to 2[(1−γ)σ²+(1+γ)κ²], presuming the estimand of interest is τ=μ₁−μ₀ such that r₀′=−1 and r₁′=1. Comparing to the asymptotic variance of the unadjusted estimator that yields v_{unadj}²=4σ², this demonstrates that even in the worst-case scenario where γ=−1, the semiparametric efficient estimator has the same asymptotic variance as the unadjusted estimator. In the best-case scenario where γ=1, the asymptotic variance is 4κ². γ mediates the extent to which the asymptotic variance depends on the marginal variance or the average conditional variance.

Process 100 in accordance with embodiments of the invention further includes setting (108) a desired power level. In a number of embodiments, the desired power level was set to 0.8. Significance level a was set to 0.05 in all embodiments of the invention. The asymptotic variances of the various estimators are then used in the power formula along with the desired power level to determine the sample size necessary. In many embodiments, presuming that the statistical significance of the result is assessed using a two-sided p-value cutoff p<α, the probability of a desired event occurring when in fact the true effect is τ such that

$$\hat{τ} \sim N ( τ , \frac{{v}^{2}}{n} )$$

is:

$$Power = ϕ ( {ϕ}^{- 1} ( \frac{α}{2} ) + \sqrt{n} \frac{τ}{v} ) + ϕ ( {ϕ}^{- 1} ( \frac{α}{2} ) - \sqrt{n} \frac{τ}{v} ) \qquad ( 5 )$$

where ϕ denotes the CDF of the standard normal distribution, and τ represents the treatment effect. Process 100 in accordance with embodiments of the invention determines (110) a sample size n in conjunction with an estimator of their choosing without enrolling more trial subjects than necessary to attain the desired power. Sample sizes n_{AIPW†} and n_{unadj†} are determined according to the formula:

$${n}^{†} = \{ \begin{matrix}arg {min}_{n} n \\ s . t . 1 - β < ϕ ( {ϕ}^{- 1} ( \frac{α}{2} ) + \sqrt{n} \frac{τ}{υ} ) + ϕ ( {ϕ}^{- 1} ( \frac{α}{2} ) - \sqrt{n} \frac{τ}{υ} )\end{matrix} \qquad ( 6 )$$

where n_{unadj†} represents the enrollment of the trial necessary to achieve the desired power if the unadjusted power formula was used, and n_{AIPW†} represents the enrollment necessary to achieve the desired power if the power formula was adjusted accordingly with the implementation of an AIPW estimator. Though the asymptotic variance of any chosen estimator ultimately may depend on the uncertain sampling process, this may be resolved by performing the power analysis with an estimator that must always attain a larger sampling variance than the estimator that will ultimately be utilized, but also allows for tractable estimation of the asymptotic sampling variance from a small number of interpretable population parameters.

### Estimator Design

In the context of a randomized trial, the main benefit of using an AIPW estimator (or other semiparametric efficient estimator) is that it has the smallest possible variance among reasonable estimators. As a consequence, it produces the smallest confidence intervals. The AIPW estimator is given by:

\hat{τ}=r(μ₀,μ₁) (7)

$${\hat{μ}}_{w} = \hat{𝔼} [ \frac{{W}_{w}}{{π}_{w}} ( Y - {\hat{μ}}_{w}^{( - k )} ( X ) + {\hat{μ}}_{w}^{( - k )} ( X ) ] \qquad ( 8 )$$

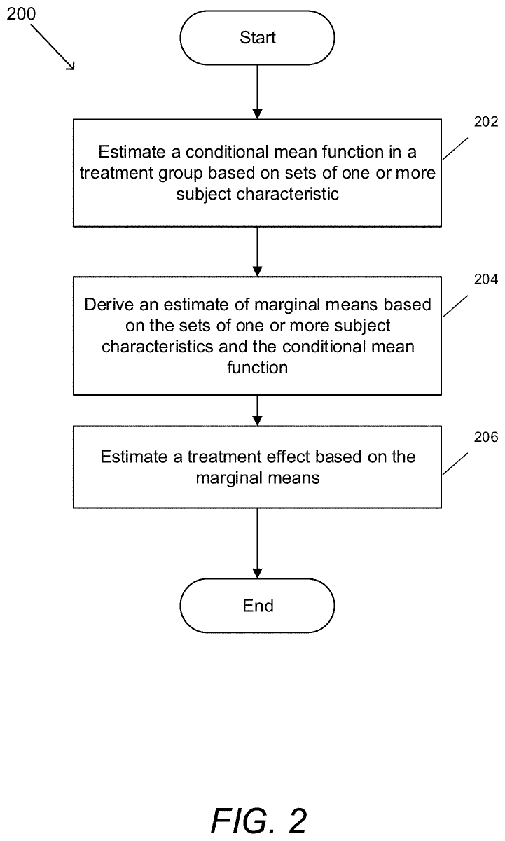

A conceptual illustration of the AIPW estimator in accordance with embodiments of the invention is shown in FIG. 2. Process 200 estimates (202) a conditional means function \hat{μ}_{w}(X) for each treatment group based on the sets of one or more subject characteristics and a machine learning model. This produces estimated versions of the true, unknown, conditional means \hat{μ}_{w}(X)= [symbol] [Y|X,W=w]. Process 200 further derives (204) an estimate of marginal means \hat{μ}_{w} with subject characteristics (X, W, Y) and temporarily ignoring (−κ) superscripts. Process 200 estimates (206) a treatment effect c based on estimated marginal means. Though it must be noted, additional assumption needed to be made in order for asymptotic properties to hold while (−κ) superscripts are ignored. To prevent additional assumptions, conditional means must be cross-estimated from the subject characteristics. This requires a splitting of subject characteristic data into K non-overlapping folds and fit K different models for \hat{μ}_{w}(X), each excluding data from one of the folds, which is denoted by \hat{μ}_{w}^{(-k)}(X). Models are trained without data of the kth fold in order to make predictions for the kth fold, corresponding to predicting the eventual treatment effect of the trial subject. This avoids any unknowing overfitting of the machine learning models, and any conclusions based on the AIPW estimator are agnostic to the specific machine learning model that may be used.

As discussed above in the estimating (104) of population parameters, function r in the AIPW estimator defines the treatment effect from the true mean outcomes μ_{w}= [symbol] [Y_{w}]. By letting

$${r}_{w}^{′} = \frac{\partial r}{\partial {μ}_{w}} ( {μ}_{0} , {μ}_{1} ) ,$$

any semiparametric efficient estimator is asymptotically normal \sqrt{n}(\hat{τ}−τ) [symbol] N(0,v*²) where v*² is the efficiency bound given by:

v_{*}²= [symbol] [ϕ] (9)

ϕ=r₀′ϕ₀+r₁′ϕ₁ (10)

$${ϕ}_{w} = \frac{{W}_{w}}{{π}_{w}} ( Y - {μ}_{w} ( X ) ) + ( {μ}_{w} ( X ) - {μ}_{w} ) \qquad ( 11 )$$

### Simulation Results

Further simulation was performed to confirm whether sample sizes greater than n_{AIPW†} would indeed produce a power that is higher than the desired power. Additionally, simulations were performed to ascertain the exact increase in power level due to the increases in sample sizes among estimators including the cross-fitted AIPW estimator, analysis of covariance (ANCOVA) estimator, as well as an “oracle” AIPW estimator with access to true conditional means functions μ_{w}(X). Simulations were performed in four different scenarios including linear and nonlinear conditional means functions and the presence or absence of treatment effect heterogeneity. In all cases, the distribution of covariates P(X) was a 10-dimensional uniform random variable in the prism [−1,1]¹⁰. P(Y_{W}|X) were of a Gaussian quadratic-mean form [symbol] (aX^{T} [symbol] X+bX^{T} [symbol] +c,1). Parameter a controls the degree of non-linearity, where a linear case is represented by a=0. Treatment effect heterogeneity refers to a situation where a or b is different for P(Y₀|X), and P(Y₁|X). Parameter c is modified in each scenario such that the average treatment effect became 0.

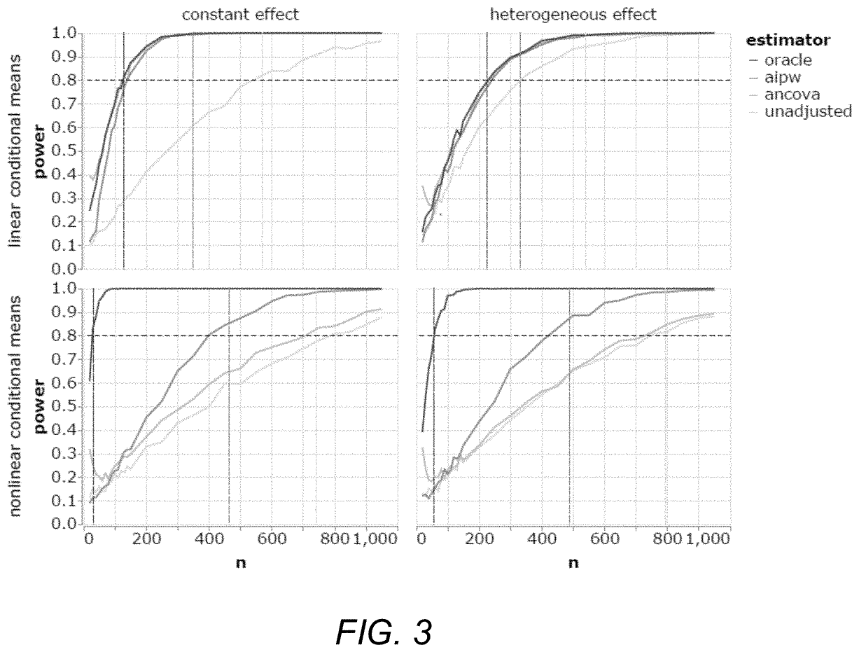

FIG. 3 illustrates empirical powers of the simulations of the four scenarios with four different estimators in accordance with embodiments of the invention. The results demonstrate trials designed with AIPW estimator may attain power greater than 80% with increased enrollment greater than n_{AIPW†}. Potential savings of approximately 35% was also observed in the simulations due to the smaller sample size. AIPW estimators also outperformed its ANCOVA and unadjusted counterparts in the non-linear cases, suggesting an opportunity to improve the quality of the conditional means modeling in the AIPW estimator.

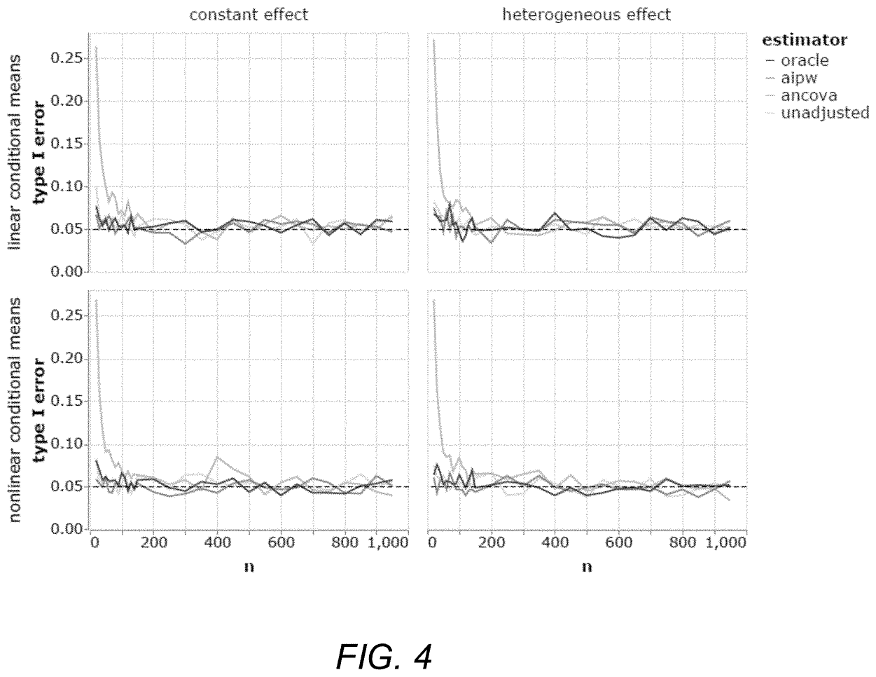

FIG. 4 illustrates the type I error rates across the four scenarios for the four estimators. The AIPW estimator is able to control type I error in large samples.

Processes disclosed herein were further tested in a clinical trial through the Alzheimer's Disease Cooperative Study. Population parameters were estimated from subject characteristics generated from 6,919 early-stage Alzheimer's patients provided by the Alzheimer's Disease Neuroimaging Initiative (ADNI) and the Critical Path for Alzheimer's Disease (CPAD). Sample size estimation with an unadjusted estimator yielded a required enrollment of n_{unadi†}=272 subjects to produce a power level of over 80% at a significance level of 0.05, whereas sample size estimation with a semiparametric efficient estimator yielded n_{AIPW†}=243. Sample size calculation with an AIPW estimator resulted in approximately 10% savings.

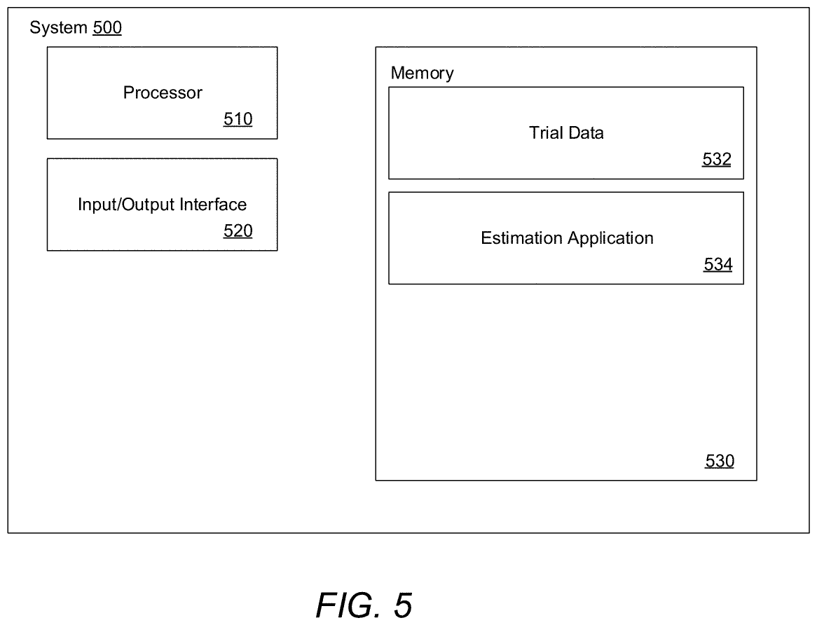

An example of a computing system that processes described above can be implemented on in some embodiments of the invention is illustrated in FIG. 5. System 500 includes an input/output interface 520 that can receive data from control arms of prior trials and registry data, and a memory 530 to store the data from control arms of prior trials and registry data under an overall trial data memory 532. Processor 510 may execute the estimation application 534 to perform an estimation of sample size necessary for the desired power level in accordance with several embodiments of the invention. One skilled in the art will recognize that the computing system may exclude certain components and/or include other components that are omitted for brevity without departing from this invention.

Processor 510 can include a processor, a microprocessor, controller, or a combination of processors, microprocessor, and/or controllers that performs instructions stored in the memory 530 to manipulate trial data stored in the memory. Processor instructions can configure the processor 510 to perform processes in accordance with certain embodiments of the invention. In various embodiments, processor instructions can be stored on a non-transitory machine readable medium.

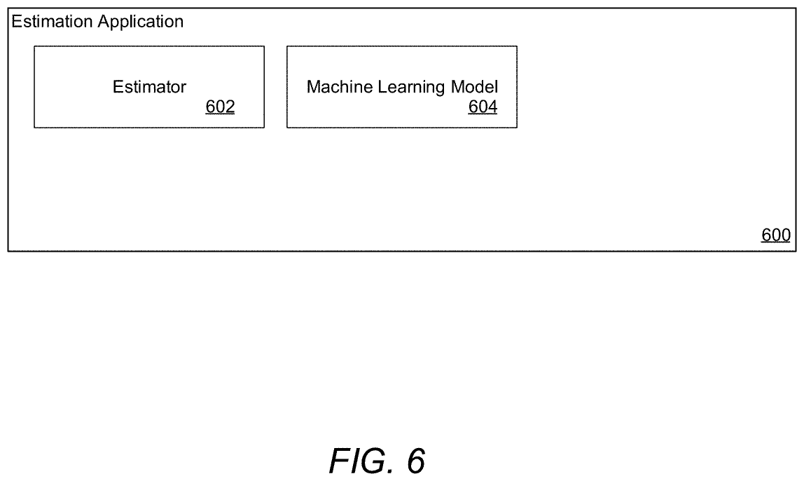

An example of an estimation application that executes instructions to estimate sample sizes necessary to attain a desired power level of a trial in accordance with an embodiment of the invention is illustrated in FIG. 6. Estimation application 600 includes an estimator 602, and a machine learning model 604. Estimator 602 in accordance with various embodiments of the invention can be used to estimate the sample size necessary to attain a desired power level of a trial. In several embodiments, the machine learning model 604 can be used to generate the subject characteristics from data from control arms of prior trials and registry data stored in the memory.

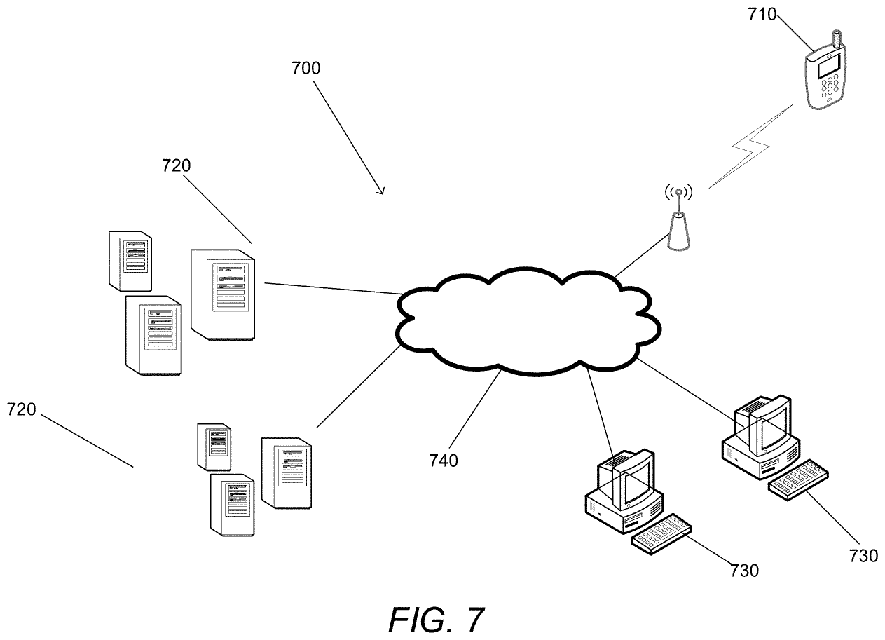

An example of a network that processes described above can be implemented on in some embodiments of the invention is illustrated in FIG. 7. Network 700 includes a communication network 740. The communication network 740 is a network such as the Internet that allows devices connected to the network 700 to communicate with other connected devices. Server systems 720 are connected to the network 740. Each of the server systems 720 is a group of one or more servers communicatively connected to one another via internal networks that execute processes that provide cloud services to users over the network 740. For purposes of this discussion, cloud services are one or more applications that are executed by one or more server systems to provide data and/or executable applications to devices over a network.

The server systems 720 are shown each having three servers in the internal network. However, the server systems 720 may include any number of servers and any additional number of server systems may be connected to the network 740 to provide cloud services. In accordance with various embodiments of this invention, a computing system that uses systems and methods that estimate sample size necessary to attain a desired power in a trial in accordance with an embodiment of the invention may be provided by a process being executed on a single server system and/or a group of server systems communicating over network 740.

Users may use personal devices 710 and 730 that connect to the network 740 to perform processes that estimate sample size necessary to attain a desired power in a trial in accordance with various embodiments of the invention. In the shown embodiment, the personal devices 730 are shown as desktop computers that are connected via a conventional “wired” connection to the network 740. However, the personal device 730 may be a desktop computer, a laptop computer, a smart television, an entertainment gaming console, or any other device that connects to the network 740 via a “wired” connection. The mobile device 710 connects to network 740 using a wireless connection. A wireless connection is a connection that uses Radio Frequency (RF) signals, Infrared signals, or any other form of wireless signaling to connect to the network 740. In the example of this figure, the mobile device 710 is a mobile telephone. However, mobile device 710 may be a mobile phone, Personal Digital Assistant (PDA), a tablet, a smartphone, or any other type of device that connects to network 740 via wireless connection without departing from this invention.

Although specific methods of designing efficient randomized trials using semiparametric efficient estimators for power and sample size calculation are discussed above, many different design methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for sample size estimation using semiparametric efficient estimators, the method comprising:
generating sets of one or more subject characteristics of a plurality of trial subjects based on data of prior trials and registry data;
estimating sets of one or more population parameters based on the sets of one or more subject characteristics;
estimating asymptotic variances of a plurality of estimators using the sets of one or more population parameters;
setting a desired power level for the trial; and
determining a sample size necessary to attain the desired power level for the trial based on the asymptotic variances and a treatment effect estimated by a semiparametric efficient estimator.

2. The method of claim 1, where estimating the treatment effect using the semiparametric efficient estimator comprises:
estimating a conditional means function in a treatment group based on sets of one or more subject characteristic data;
deriving an estimate of marginal means based on the sets of one or more subject characteristics and the conditional means function; and
estimating a treatment effect based on the marginal means.

3. The method of claim 2, where estimating the conditional means function comprises:
splitting the sets of one or more subject characteristic data into a plurality of overlapping folds;
fitting a corresponding machine learning model for each of the plurality of overlapping folds;
excluding subject characteristic data of a last of the plurality of folds; and
training the machine learning model to estimate the conditional means function by predicting subject characteristic data of the last of the plurality of folds.

4. The method of claim 1, where the sets of one or more subject characteristics include outcomes, baseline covariates, and treatment assignments.

5. The method of claim 1, where the semiparametric efficient estimator is an augmented inverse propensity weighting (AIPW) estimator.

6. The method of claim 1, where the sets of one or more population parameters may be estimated with a machine learning model in combination with the sets of one or more subject characteristics.

7. The method of claim 1, where the sets of one or more population parameters may include marginal variances, average conditional variances, and a correlation between conditional means.

8. The method of claim 1, where the semiparametric efficient estimator is a targeted maximum likelihood estimation (TMLE) estimator.

9. A non-transitory machine readable medium containing processor instructions for sample size estimation using semiparametric efficient estimators, where execution of the instructions by a processor causes the processor to perform a process that comprises:
generating sets of one or more subject characteristics of a plurality of trial subjects based on data of prior trials and registry data;
estimating sets of one or more population parameters based on the sets of one or more subject characteristics;
estimating asymptotic variances of a plurality of estimators using the sets of one or more population parameters;
setting a desired power level for the trial; and
determining a sample size necessary to attain the desired power level for the trial based on the asymptotic variances and a treatment effect estimated by a semiparametric efficient estimator.

10. The non-transitory machine readable medium of claim 9, where estimating the treatment effect using the semiparametric efficient estimator comprises:
estimating a conditional means function in a treatment group based on sets of one or more subject characteristic data;
derive an estimate of marginal means based on the sets of one or more subject characteristics and the conditional means function; and
estimating a treatment effect based on the marginal means.

11. The non-transitory machine readable medium of claim 10, where estimating the conditional means function comprises:
splitting the sets of one or more subject characteristic data into a plurality of overlapping folds;
fitting a corresponding machine learning model for each of the plurality of overlapping folds;
excluding subject characteristic data of a last of the plurality of folds; and
training the machine learning model to estimate the conditional means function by predicting subject characteristic data of the last of the plurality of folds.

12. The non-transitory machine readable medium of claim 9, where the sets of one or more subject characteristics include outcomes, baseline covariates, and treatment assignments.

13. The non-transitory machine readable medium of claim 9, where the semiparametric efficient estimator is an augmented inverse propensity weighting (AIPW) estimator.

14. The non-transitory machine readable medium of claim 9, where the sets of one or more population parameters may be estimated with a machine learning model in combination with the sets of one or more subject characteristics.

15. The non-transitory machine readable medium of claim 9, where the sets of one or more population parameters may include marginal variances, average conditional variances, and a correlation between conditional means.

16. The non-transitory machine readable medium of claim 9, where the semiparametric efficient estimator is a targeted maximum likelihood estimation (TMLE) estimator.
